(19) United States
(12) Patent Application Publication
(10) Pub. No.: US 2024/0215861 A1
(43) Pub. Date: Jul. 4, 2024

(54) Facial Recognition System and Physiological Information Generative Method

(71) Applicant: Industrial Technology Research Institute, Hsinchu (TW)

(72) Inventors: Wen-Hung Ting, Tainan City (TW); Chia-Chang Li, Pingtung County (TW)

(73) Assignee: Industrial Technology Research Institute, Hsinchu (TW)

(21) Appl. No.: 18/512,052

(22) Filed: Nov. 17, 2023

Related U.S. Application Data
(60) Provisional application No. 63/436,081, filed on Dec. 29, 2022.

(30) Foreign Application Priority Data
Aug. 8, 2023 (TW) 112129674

(51) Int. Cl. A61B 5/087; G06V 40/16; G08B 21/02

(52) U.S. Cl. CPC A61B 5/0878; G06V 40/168; G08B 21/0202

(57) ABSTRACT

A facial recognition system and a physiological information generative method are disclosed. The facial recognition system includes a visible light sensor, a thermal imaging sensor, and a processor. The generative method is executed by the processor, using a real-time object detection algorithm. The generative method includes receiving one or more current visible and thermal images, and identifying the nasal area in the current thermal images when a face region in the current visible images is not identifiable by the real-time object detection algorithm in the processor; determining in the processor that respiratory information is abnormal according to the cycles of exhalation and inhalation, as detected through brightness changes in the nostril area; and notifying abnormal respiratory information.

### CROSS-REFERENCE TO RELATED APPLICATIONS

This application claims the priority benefits of U.S. provisional application Ser. No. 63/436,081 filed on Dec. 29, 2022, and Taiwan application serial no. 112129674, filed on Aug. 8, 2023. The entirety of each of the above-mentioned patent applications is hereby incorporated by reference herein and made a part of this specification.

### TECHNICAL FIELD

This disclosure relates to an image recognition system and an image identification method thereof. This disclosure also relates to a facial recognition system and a physiological information generative method thereof.

### BACKGROUND

An image recognition system, such as webcam, monitor, camera, and the like, can identify a human shape as a region of interest for the human's health and safety. For instance, a webcam in a hospital can be used to monitor an infant for any abnormalities in the infant's physical condition, especially if the infant is being monitored at home. After her baby is born, a first-time mother returns home to care for her baby and needs to remain attentive to any abnormalities in the infant's physical condition. When the infant has a fever or irregular breathing, or the infant suddenly died from suffocation due to milk regurgitation or turning over. Therefore, the first-time mother can use the webcam to monitor the infant's physical condition. The webcam can locate the relative positions of the facial features within a face shape through one or more visible light images, thereby recognizing whether mouth and nose area of the infant are covered by one or more covers, such as blankets, pillows, clothing, or stuffed toys. The thermal imaging camera can measure the body temperature of the infant through one or more thermal images. The contact sensor includes a pulse sensor and an accelerometer. The pulse sensor can measure the heart rate of the infant. The accelerometer can measure the respiratory rate of the infant. Additionally, the webcam in a hospital also can be used to monitor elderly persons, patients, and kids' for their health and safety. Also, the webcam can be place in any proper place, either indoors or outdoors.

However, the webcam cannot identify covers with irregular shapes, positioned at any angle, that obscure parts of the facial features in a face region of the infant. The webcam also cannot identify the abnormal temperature of the infant on one or more visible images. Additionally, the thermal imaging camera identifies the temperature of the infant as abnormal, but in reality, the webcam identifies the infant wearing more clothes as normal. Also, the contact sensor cannot remain on the infant's skin for extended periods while measuring the heart rate or respiratory rate.

### SUMMARY

The facial recognition system includes a detector and a host computer. The detector includes a visible light sensor and a thermal imaging sensor. The visible light sensor within a target area is configured to capture one or more current visible images. The thermal imaging sensor within the target area is configured to capture one or more current thermal images. The host computer is coupled to the detector and configured to execute a real-time object detection algorithm to notify abnormal respiratory information when the host computer determines a nasal area in the current thermal images is abnormal, wherein the real-time object detection algorithm is configured to identify a face region in the current visible and thermal images, When the face region in the current visible images is not identifiable, the real-time object detection algorithm identifies the nasal area of the face region in the current thermal images, and the host computer generates the abnormal respiratory information according to the cycles of exhalation and inhalation, as detected through brightness changes in the nostril area.

The physiological information generative method executed a real-time object detection algorithm by the processor. The physiological information generative method includes receiving one or more current visible and thermal images; identifying the nasal area in the current thermal images when a face region in the current visible images is not identifiable by the real-time object detection algorithm of the processor; determining that respiratory information is abnormal according to the cycles of exhalation and inhalation, as detected through brightness changes in the nostril area; and notifying abnormal respiratory information.

### BRIEF DESCRIPTION OF THE DRAWINGS

FIG. 1 shows a schematic view of a facial recognition system according to one embodiment of the present disclosure.

FIG. 2 shows a schematic view of a configuration related to FIG. 1.

FIG. 3A shows a schematic view of another configuration related to FIG. 1.

FIG. 3B shows a schematic view of another configuration related to FIG. 1.

FIGS. 4A-4F shows a generative method for physiological information according to one embodiment of the current thermal images.

FIG. 5 shows an operational scenario diagram of the forehead area identification according to one embodiment of the current visible images.

FIG. 6 shows a signal waveform diagram of the forehead area identification related to FIG. 5.

FIG. 7 shows the current visible images of infants without obstructions.

FIG. 8 shows the current visible images of infants with obstructions.

FIG. 9 shows an operational scenario diagram of the infant's nostril identification in exhalation according to one embodiment of the current thermal images.

FIG. 10 shows an operational scenario diagram of the infant's nostril identification in inhalation according to one embodiment related to FIG. 11.

FIG. 11 shows an operational scenario diagram of the adult's nostril identification in exhalation according to one embodiment of the current thermal images.

FIG. 12 shows an operational scenario diagram of the adult's nostril identification in inhalation according to one embodiment related to FIG. 13.

FIG. 13 shows an operational scenario diagram of the forehead temperature identification according to one embodiment.

### DETAILED DESCRIPTION

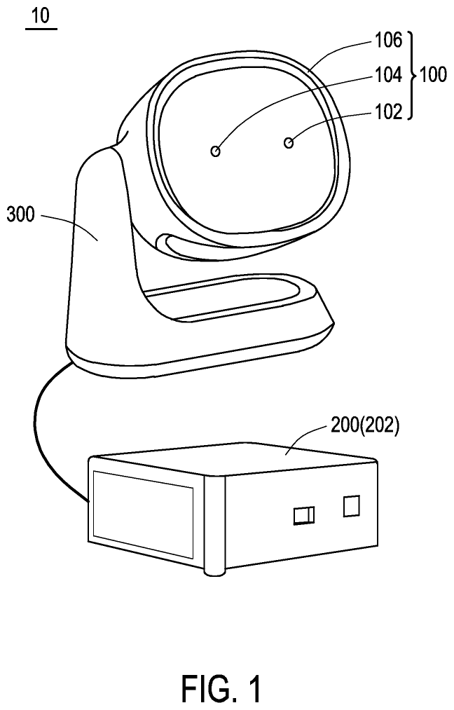

Please refer to FIG. 1, a facial recognition system 10 includes a detector 100, a host computer 200, and a stand 300. The detector 100 that is supported by the stand 300 is coupled to the host computer 200 either by wires or wireless unit. For example, the detector 100 and the host computer 200 are coupled by wires or via the internet to transmit and receive the captured images and physiological information for further processing or storage. The stand 300 includes a three-axis mechanism that can tilt the detector 100 upward or downward to face individuals. Additionally, in one embodiment of the stand 300 further includes a six-axis mechanism that can rotate the detector 100 to ensure both the visible light sensor 102 and the thermal imaging sensor 104 face individuals, such as an infant, an adult, or an elder. The visible light sensor 102 and the thermal imaging sensor 104 are disposed on the detector 100.

In one embodiment, the detector 100 can be positioned within the target area to face individuals, such as an infant, adult, or elder indoors. For example, the detector 100 can be positioned in a room to face an infant's face. In an example, the detector 100 can capture the infant's face images. The detector 100 includes a visible light sensor 102 and a thermal imaging sensor 104, which are disposed on a first housing 106. The visible light sensor 102 is configured to capture one or more current visible images (VSI) within the target area. The thermal imaging sensor 104 is configured to capture one or more current thermal images (THI) within the target area.

The visible light sensor 102 and the thermal imaging sensor 104 can detect the same individual's face. For example, a plurality of visible images (VSI), one or more covers with irregular shapes obscure the individual's nose and mouth. The cover can also be positioned at any angle that obscures the infant's nose and mouth. A plurality of thermal images (THI), brightness of the individual's nose is changed as an infant exhales and inhales.

The visible light sensor 102 can be a Charge-coupled Device (CCD) or a CMOS to constantly detect the plurality of visible images (VSI) for a period of time, which can be either continuous or discontinuous. For example, the CMOS can detect a series of current continuous the visible images (VSI) that capture the brightness of the forehead area as an infant exhales and inhales.

In one embodiment, the visible light sensor 102 can capture the current visible images within an illuminance range from about 50 lux to below 1000 lux, a real-time object detection algorithm can determine that the current visible images are identifiable based on illuminance.

In one embodiment, when the maximum capture distance of the visible light sensor 102 increases from below 40 cm to below 60 cm, the real-time object detection algorithm can determine that the current visible images are identifiable based on the distance.

In one embodiment, the visible light sensor 102 can be coupled with an active light source, which is used to increase the brightness in the room.

The thermal imaging sensor 104 can serve as an infrared thermal imager to detect a plurality of thermal images (THI) for a period of time, which can be either continuous or discontinuous. For example, the infrared thermal imager can detect a series of continuous the thermal images (THI) to capture a heat gradient distribution of each thermal image. The infrared thermal imager includes one or more infrared sensors, one or more optical lenses, and an imaging processor (the drawings not show). The infrared sensor can capture an infrared radiation and generate an infrared signals when the infrared radiation is emitted by objects. The infrared sensor can be thermocouples, thermopiles, optical arrays, and the like. The imaging processor can convert the infrared signals into thermal images (THI). The optical lens can focus the infrared radiation from the object's surface onto the radiation sensor. For example, the imaging processor generates a series of thermal images (THI) that capture brightness changes of the infant's nose.

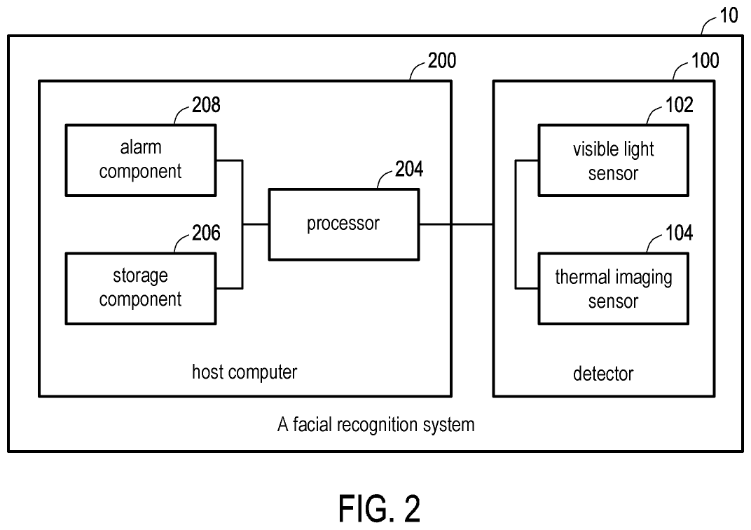

Please refer to FIG. 1 and FIG. 2, the host computer 200 includes a second housing 202, a processor 204, a storage component 206, and an alarm component 208. The processor 204, the storage component 206, and the alarm component 208 are disposed in the second housing 202.

The processor 204 is coupled to the storage component 206 and the alarm component 208. For example, the processor 204 can be a Central Processing Unit (CPU), Graphics Processing Unit (GPU), Micro Processing Unit (MPU), Digital Signal Processor (DSP), Microcontroller Unit (MCU), and so on.

In one embodiment, when the visible light sensor 102 captures the current visible images, and the thermal imaging sensor 104 captures the current thermal images, the processor 204 executes a generative method for physiological information S100 using a real-time object detection algorithm. The real-time object detection algorithm includes deep learning algorithms such as Convolutional Neural Networks (CNN), Recurrent Neural Networks (RNN), Cross Stage Partial Networks (CSPNet), Region-based Convolutional Neural Networks (R-CNN), and You Only Look Once (YOLO), and the like. For example, You Only Look Once (YOLO) in the realm of computer science refers to a real-time object detection algorithm that can detect multiple objects in images or video frame. YOLO includes CSPDarknet 53, which is formed by combining Convolutional Neural Network (CNN), Densely Connected Convolutional Network (DenseNet), and Cross stage partial network (CSPNet).

When the processor 204 executes YOLO, YOLO can be trained to recognize face regions in both the current visible images (VSI) and the current thermal images (THI), including infant images and adult images and so on. In each face region. YOLO can identify specific areas such as the forehead, eyes, nostrils, mouth, eyebrows. The default YOLO can scale one or more a series of continuous thermal images (THI) to the same size, and then divides the series of continuous thermal images (THI) into a plurality of grid cells (Grid Cell). Each Grid Cell can predict a plurality of bounding boxes simultaneously and generate confidence scores for each bounding box. The confidence scores reflect trust level of YOLO in the nostril area delineated by the bounding box and the prediction accuracy of this bounding box. Next, YOLO can process convolution and residual operations on the series of continuous thermal images (THI) through a Darknet-53 and a Feature Pyramid Network (FPN). Thus, YOLO can output feature maps of three different scales, and predict physiological information corresponding to the face region in the series of continuous thermal images (THI). After identifying the face region in both the current visible images (VSI) and the current thermal images (THI), the real-time object detection algorithm generates physiological information, which includes heart rate information, respiratory information, and temperature information and so on.

The storage component 206 is configured to store the current visible images (VSI), the current thermal images (THI), and the real-time object detection algorithm. The storage component 206 can be non-volatile memories such as hard drives, optical discs, portable hard drives, flash memory, solid-state drives, etc., and volatile memories such as dynamic random-access memory (DRAM) and static random-access memory (SRAM).

The alarm component 208 can be a buzzer or indicator light. When the real-time object detection algorithm in the processor 204 generates the abnormal respiratory information, the processor 204 can activate the alarm component 208. In one embodiment, the alarm component 208 can disposed in the host computer 200. In another embodiment, the alarm component 208 can disposed in the first housing 106 of the detector 100.

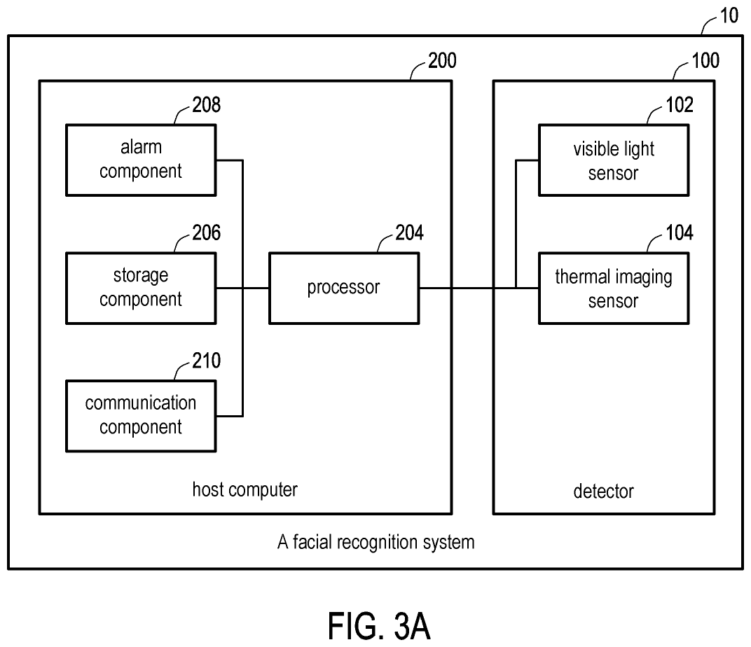

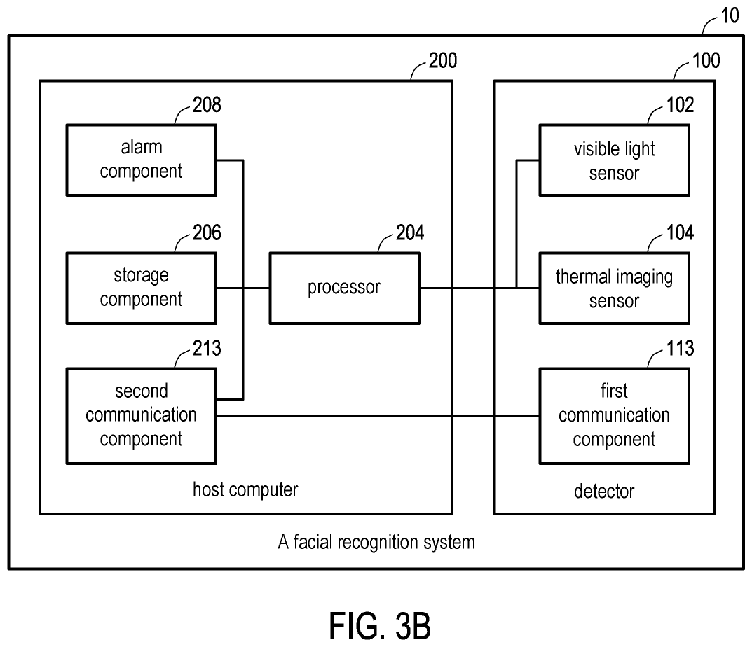

Please refer to FIG. 3A, in one embodiment, the facial recognition system 10 further includes the communication component 210, which is disposed in the second housing 202 and coupled to the processor 204. The communication component 210 is configured to transmit and receive physiological information through a wired or wireless, network connection, or via an Access Point (AP). For example, the communication component 210 can use wireless technologies, such as WiFi, Bluetooth, and others. The communication component 210 can transmit physiological information to a user equipment, or receive the physiological information from the user equipment. The user equipment can be smart phones, pads, servers, and so on.

Please refer to FIG. 3B, in one embodiment, the facial recognition system 10 further includes a first communication component 113 and a second communication component 213. For example, the first communication component 113 is disposed in the detector 100. The second communication component 213 is disposed in the host computer 200, and coupled to the processor 204. When the visible light sensor 102 and the thermal imaging sensor 104 detect the same individual's face information, the first communication component 211 transmits the individual's face information to the second communication component 113. The individual's face information includes the face region in the current visible images (VSI) and the current thermal images (THI), such as a forehead area, an eyebrow area, an eye area, a nostril area, and a mouth area.

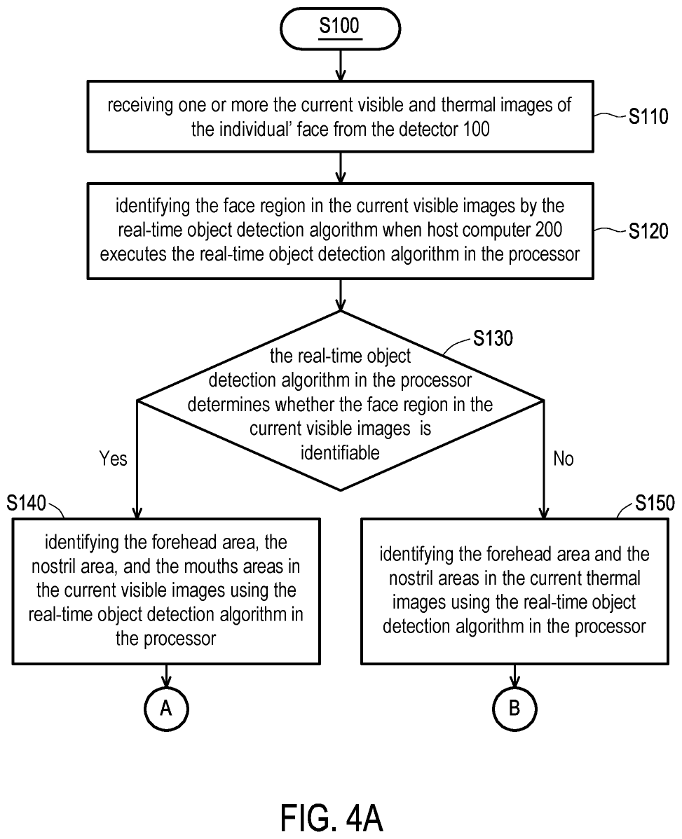

Please refer to FIGS. 4A-4F, the physiological information generative method S100 is executed by the facial recognition system 10. The physiological information generative method S100 uses the real-time object detection algorithm in the processor 204 to identify individual's physiological information based on the face region in the current visible images and the current thermal images (VSI, THI). The face region includes the forehead, eyebrow, eye, nostril, and mouth areas. The physiological information includes heart rate, respiratory information, and temperature information and so on.

In one embodiment, when the detector 100 faces the individual' face, the detector 100 captures the current visible images (VSI) and the current thermal images (THI) of the individual's face. Then, the host computer 200 executes YOLO, such that YOLO identifies the individual's face region. Next, the host computer 200 generates the physiological information corresponding to each identified area of the face region. Thus, the host computer 200 determines whether the physiological information is abnormal by comparing the generated physiological information with reference information. If YES, the host computer 200 transmits a notification to a user equipment or activates the alarm component 208 alarms; if NO, the host computer 200 continuously records and stores the normal physiological information in the storage component 206. The reference information can be an individual's normal value range, such as a normal temperature range, heart range, and respiratory range.

As shown in FIG. 4A, when identifying the face region, YOLO identifies the face region in the current visible images (VSI), and then in the current thermal images (THI) in order. Namely, YOLO determines whether the face region in the current visible images (VSI) is identifiable, if YES, YOLO further identifies the forehead, nostril, and mouth areas; if NO, YOLO identifies the face region in the thermal visible images (THI).

For example, in step S110, the host computer 200 receives the current visible and thermal images (VSI, THI) of the individual's face from the detector 100. In step S120, the host computer 200 executes the real-time object detection algorithm, such that the real-time object detection algorithm in the processor 204 identifies the face region in the current visible images (VSI). Next, in step S130, the real-time object detection algorithm in the processor 204 determines whether the face region in the current visible images is identifiable. If YES, the generative method proceeds to step S140, which includes identifying the forehead area. the nostril area, and the mouths area in the current visible images using the real-time object detection algorithm in the processor 204. If No, the generative method proceeds to step S150, which includes identifying the forehead area and the nostril area in the current thermal images using the real-time object detection algorithm in the processor 204.

Before executing step S130, YOLO, executed by the host computer 200, can be trained through the first training data. The first training data includes visible images (VSI) and thermal images (THI) of adults or infants because it is challenging to obtain the plurality of visible images (VSI) and thermal images (THI) of actual infants and young children who are not breathing or have their mouths and noses covered. For example, 15 individuals' thermal images (THI) are provided from volunteer. 14,089 infants' thermal images (THI) are provided from nurseries, wherein 8,062 of 14,089 thermal images (THI) are used for labelling the nasal area, and 6,027 thermal images (THI) are used for testing the accuracy. Then, overly similar images were removed from the first training data, such that 809 of 14,089 thermal images (THI) are left. Therefore, YOLO can identify each area of the face region as a region of interest.

When the host computer 200 executes the real-time object detection algorithm, the real-time object detection algorithm applies a neural network to all of the current visible images, such that the neural network divides each current visible image (VSI) into a grid and predicts bounding boxes and probabilities for each section in one evaluation.

As show in FIGS. 4A-4C and 5-6, the process of the heart rate identification in the current visible images (VSI) includes steps S141-S148 described as below.

In step S141, the real-time object detection algorithm in the processor 204 determines whether the forehead area, the nostril area, and the mouths area in the current visible images is identifiable. If YES, the generative method proceeds to step S142 or S160. If No, the generative method proceeds to step S150 again. Step S160 refers to the process of nostril and mouth area identification in the current visible images (VSI) and will be described later in detail.

In step S142, the real-time object detection algorithm in the processor 204 identifies the brightness changes of the forehead area. Next, in step S143, according to the brightness changes of pixels in the forehead area, the host computer 200 extracts Photoplethysmography (PPG) signals of the forehead area. Next, in step S144, the host computer 200 transforms the Photoplethysmography (PPG) signals from the time domain into the frequency domain. Next, in step S145, according to a spectrum of the frequency domain, the host computer 200 extracts the peak frequency as the heart rate information. Next, in step S146, the host computer 200 determines whether the heart rate information is normal according to a normal range. If YES, the generative method proceeds to step S147, which includes recording and storing the normal heart rate information. If No, the generative method proceeds to step S148, which includes notifying the abnormal heart rate information. Additionally, under consistent lighting and minimal head moment, a stable heart rate information can be measured within about 15 seconds by the host computer 200 using the real-time object detection algorithm.

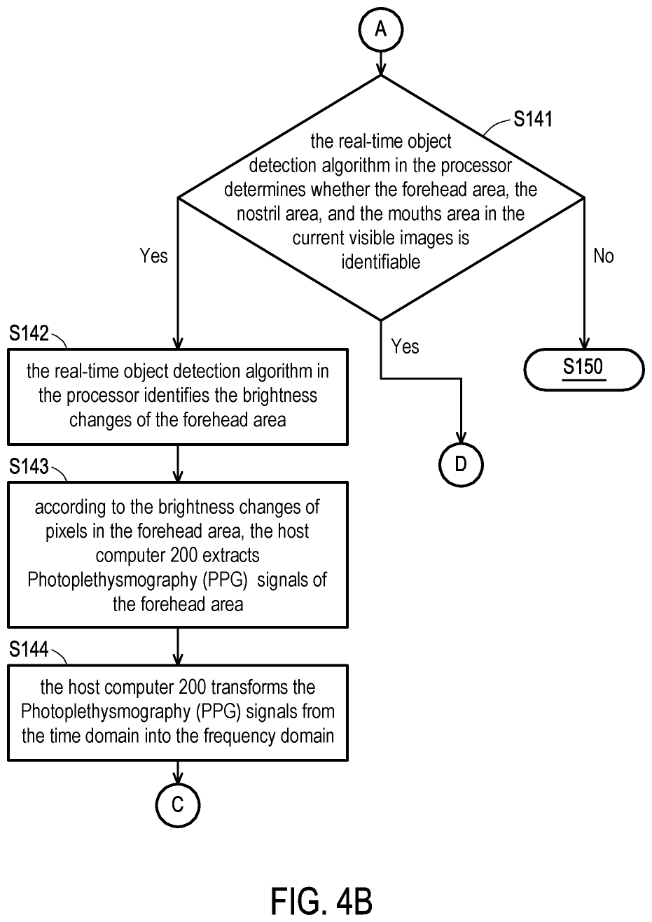

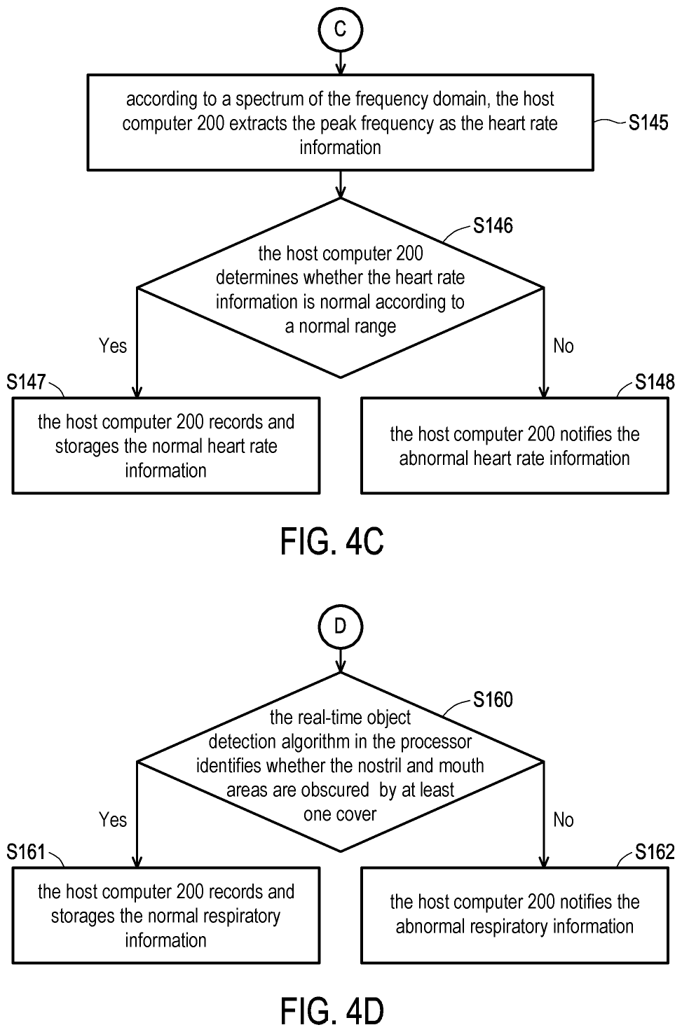

As shown in FIGS. 5-6, in one embodiment, first, YOLO can measure the heart rate information in time domain through high-pass filter. For example, YOLO uses the window 20 to extract brightness information (e.g., pixels) of the forehead area in one or a series of current continuous visible images (VSI), and then computes an average value based on the brightness information to be a constant brightness value. Next, YOLO, through time series analysis, calculates average brightness (e.g., average pixels) over a certain period to be a reference value. Thus, YOLO subtracts the reference value from the current average brightness obtained at that moment to get a relative brightness value, which can filter out low-frequency components and be transformed into frequency domain function by Fast Fourier Transformation (FFT). Also, the reference value can be dynamically updated based on varying certain periods and brightness information of the current visible images (VSI).

As shown in FIGS. 4A-4D and 7-8, the process of the process of nostril and mouth area identification in the current visible images (VSI) includes steps S160-S162 described as below.

In step S160, the real-time object detection algorithm in the processor 204 identifies whether the nostril and mouth areas are obscured by at least one cover, wherein the real-time object detection algorithm in the processor 204 can identify one or more covers with irregular shapes, positioned at any angle, that obscure both a nasal area and a mouth area of one or more current visible images (VSI). If YES, the generative method proceeds to step S161, which includes recording and storing the normal respiratory information. If No, the generative method proceeds to step S162, which includes notifying the abnormal respiratory information.

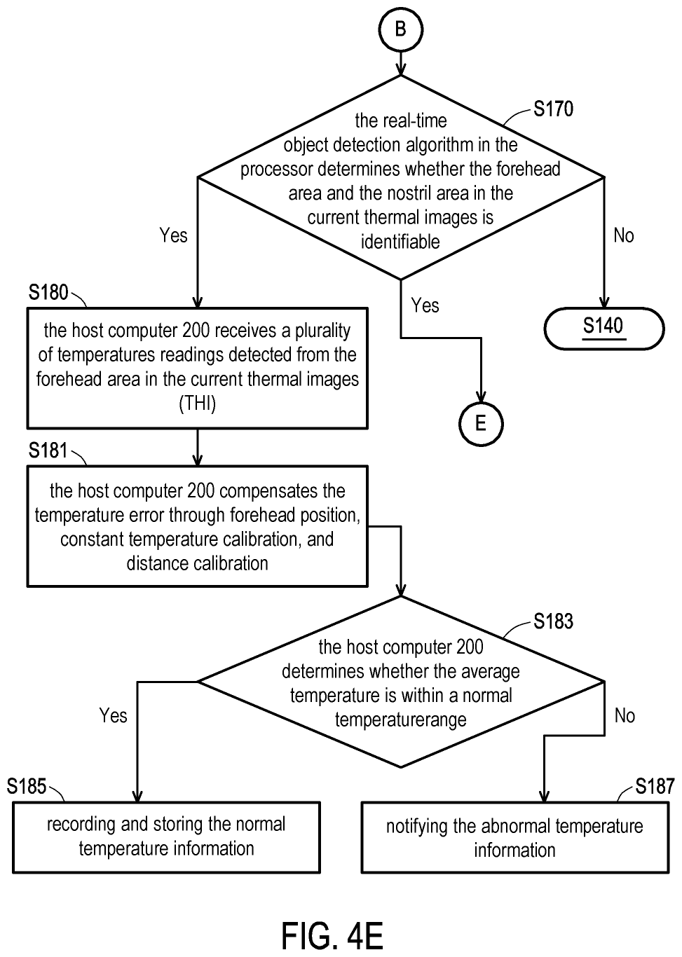

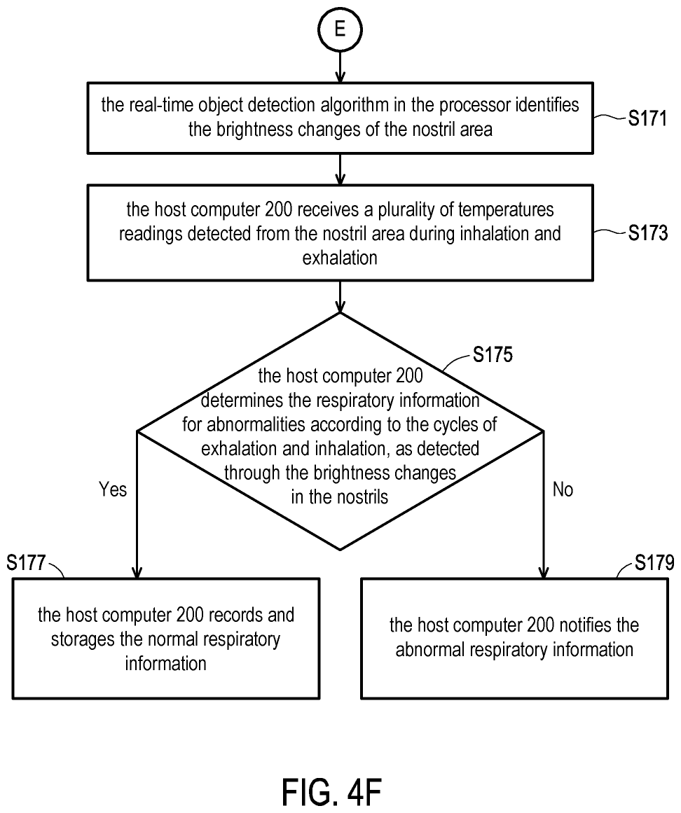

Before that, the real-time object detection algorithm in the processor 204 can be trained to identify one or more covers that obscure infant's nose and mouths in the plurality of visible images (VSI) through a second training data as shown in FIG. 7 and FIG. 8. Each cover has an irregular shape positioned at any angle. The second training data includes 2,607 infants' visible images (VSI) derived from various sources on the internet, wherein 908 of 2,607 visible images (VSI) appear both the nasal and the mouth areas without any obstructions as shown in FIGS. 7, and 772 of 2,607 visible images (VSI) appear both the nasal and the mouth areas with obstructions as shown in FIG. 8. Overly similar images were removed from the second training data.

After the training is finished, the real-time object detection algorithm in the processor 204 can be test its accuracy using a testing dataset from the internet or volunteers. The testing dataset includes 232 infant's visible images (VSI), 169 of 232 visible images (VSI) appear both the nasal and the mouth areas without any obstructions, and 66 of 232 visible images (VSI) appear both the nasal and the mouth areas with obstructions. Upon statistics, another experimental data can be presented in Table 1 as below.

TABLE 1

| | | | Error | | | error | | |
|---|---|---|---|---|---|---|---|---|
| | | | count of | Error | Omission | rate of | omission | |
| | | | normal | count of | count of | normal | rate of | |
| | unobscured | obscured | faces | obscured | obscured | faces | obscured | Accuracy |
| Item | count (P) | count (N) | (A) | faces (C) | faces (D) | (E) | faces (F) | (1-E-F) |
| Test 1 | 169 | 66 | 3 | 2 | 0 | 1.28% | 0.85% | 97.87% |

As shown in Table 1, the error rate of normal faces (E) is the Error count of normal faces (A) divided by the sum of the Unobscured Count (P) and the Obscured Count (N); the omission rate of obscured faces (F) is the sum of the Error count of obscured faces (C) and the Omission count of obscured faces (D) divided by the sum of the Unobscured Count (P) and the Obscured Count (N); the Accuracy Rate is 1 minus the sum of the error rate of normal faces (E) and omission rate of obscured faces (F).

In one embodiment, the communication component 210 can transmit the normal heart rate information to a user equipment. The user equipment can be smart phones, pads, servers, and so on.

Pleas refers to FIGS. 4A, 4E, 4F, and 9-12, the process of the nostril area identification in the thermal images (THI) includes steps S150, S170-S179 described as below in detail.

When the host computer 200 executes the real-time object detection algorithm, the real-time object detection algorithm can be trained to identify the face region in the current thermal images based on 14,089 infants' thermal images (THI). For example, YOLO applies a neural network to all of the current thermal images (THI), such that the neural network divides each current thermal image (THI) into a grid and predicts bounding boxes and probabilities for each section in one evaluation. Then, YOLO can determine whether the forehead area and the nostril area in the current thermal images is identifiable after the training is finished. That is, the real-time object detection algorithm in the processor 204 determines whether the forehead area and the nostril area in the current thermal images is identifiable, in step S170.

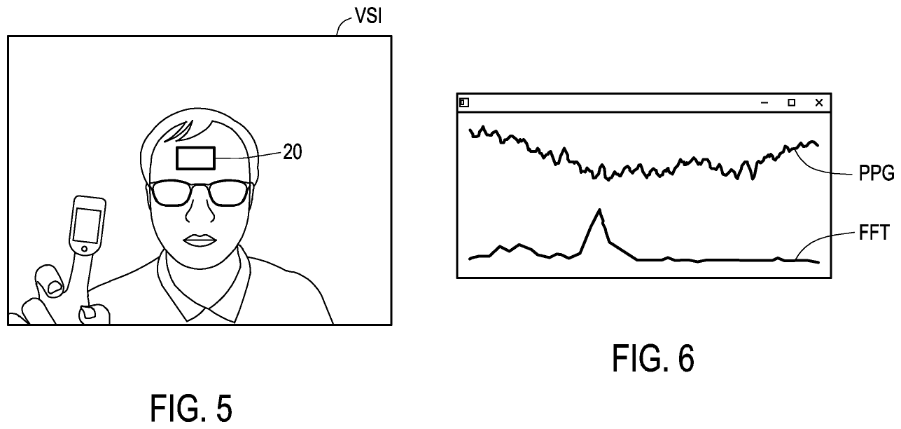

As shown in FIGS. 9 -10, in one embodiment, YOLO can identify the nostril area in the current thermal images (THI) when the detector 100 faces the infant and detects the infant's temperature. Then, YOLO use the Darknet framework to detect and count the cycles of exhalation and inhalation through the nostrils.

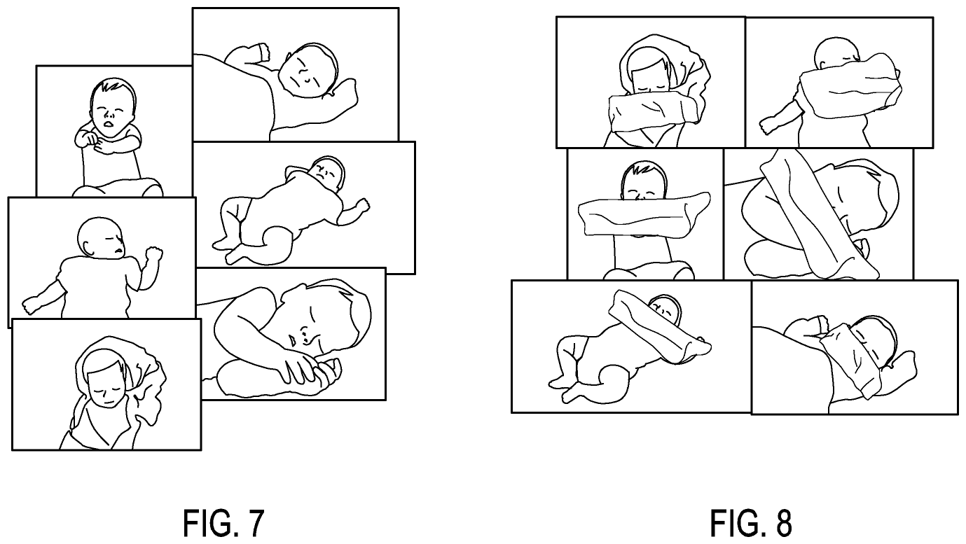

As shown in FIG. 4F, the real-time object detection algorithm in the processor 204 identifies the brightness changes of the nostril area, in step S171. Next, the host computer 200 receives a plurality of temperatures readings detected from the nostril area during inhalation and exhalation, in step S173. When exhaling, the temperature of the nostrils is slightly similar to the normal human body temperature as shown in FIG. 9. When inhaling. the temperature of the nostrils is slightly cooler than the normal human body temperature as shown in FIG. 10. Thus, the host computer 200 can determine whether the infant's respiratory information is abnormal by comparing the temperature difference of the nostrils with a normal respiratory standard. Thus, when the brightness in the nostrils remains unchanged during inhalation and exhalation for a default time period, the host computer 200 determines that the respiratory information is abnormal. Namely, the host computer 200 can determine whether the respiratory information is abnormal according to the cycles of exhalation and inhalation, as detected through the brightness changes in the nostrils, in step S175. If YES. the generative method proceeds to step S177, which includes recording and storing the normal respiratory information. If No, the generative method proceeds to step S179, which includes notifying the abnormal respiratory information.

For example, when breathing, a normal infant inhales and exhales 20 to 60 times per minute. The normal respiratory standard can be one inhalation followed by one exhalation every 3-5 seconds. If an infant's nostrils does not complete one inhalation followed by one exhalation within 3-5 seconds, their breathing is considered abnormal. That is, if the temperature difference during one respiratory cycle (one inhalation followed by one exhalation) remains unchanged for at least three seconds, their breathing is considered abnormal.

Upon statistics, another experimental data can be presented in Table 2 as below. Next, YOLO generates the respiratory information.

TABLE 2

| | | | Error | Omission | | | |
|---|---|---|---|---|---|---|---|
| | | simu- | count of | count of | | omis- | |
| | Normal | late | normal | simulate | error | sion | Accu- |
| | breaths | apnea | breaths | apnea | rate | rate | racy |
| | (P) | (N) | (A) | (B) | (E) | (F) | (1-E-F) |
| Test 1 | 100 | 20 | 1 | 0 | 0.83% | 0.00% | 99.17% |
| Test 2 | 100 | 17 | 0 | 1 | 0.00% | 0.85% | 99.15% |
| Test 3 | 100 | 39 | 0 | 1 | 0.00% | 0.72% | 99.28% |
| Avg. | 100 | 25.33 | 0.33 | 0.66 | 0.28% | 0.52% | 99.2% |

As shown in Table 2, the error rate (E) is the error count of normal breaths (A) divided by the sum of normal breaths (P) and simulate apnea (N). The omission rate (F) is the number of Not count of simulate apnea (B) divided by the sum of normal breaths (P) and simulate apnea (N). The accuracy is 1 minus the sum of the error rate (E) and the omission rate (F).

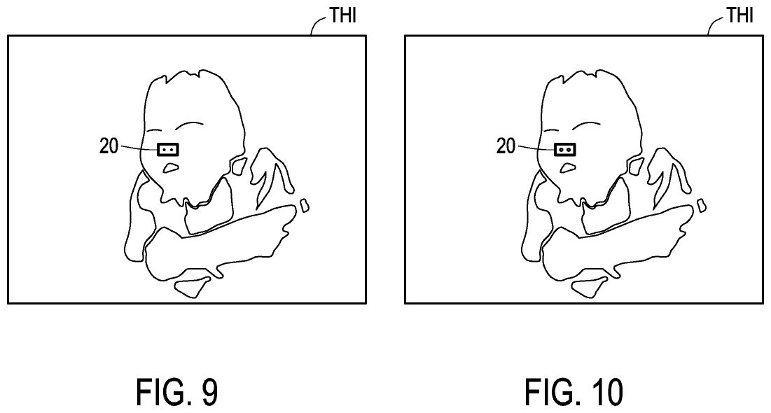

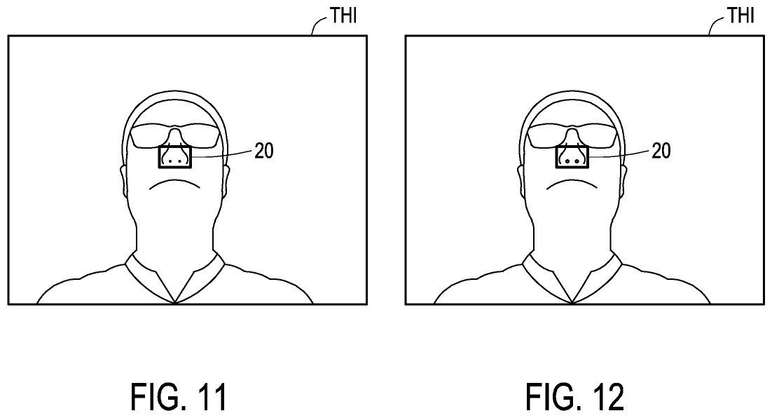

As shown in FIGS. 11-12, in one embodiment, the real-time object detection algorithm in the processor 204 can be trained based on a data, 15 thermal images (THI) of an adult, to classify the series of continuous thermal images (THI) into two types: the nostril during inhalation and the nostril during exhalation. Then, the real-time object detection algorithm in the processor 204 can be trained to identify the nostril area in the current thermal images (THI), using the individual's breaths per minute (BPM). When receiving brightness changes in the nostril between inhalation and exhalation, the host computer 200 converts one respiratory cycle (one inhalation followed by one exhalation) into the individual's breaths per minute (BPM). Thus, the host computer 200 can determine whether the adult's respiratory information is abnormal by comparing the converted breaths per minute (BPM) with the criteria of the individual's breaths per minute (BPM). Generally, the criteria of the individual's breaths per minute (BPM) illustrate as follow: newborns breathe about 40-50 times per minute; infants breathe about 30-40 times per minute; children breathe about 20-30 times per minute. If the converted BPM is above or below the criteria of the individual's breaths per minute (BPM), it can be considered as abnormal. Namely, the host computer 200 can determine whether the respiratory information is abnormal according to the cycles of exhalation and inhalation, as detected through the brightness changes in the nostrils, in step S175. If YES, the generative method proceeds to step S177, which includes recording and storing the normal respiratory information. If No, the generative method proceeds to step S179, which includes notifying the abnormal respiratory information.

As shown in FIGS. 11-12, when exhaling, the temperature of the nostrils is slightly similar to the normal human body temperature as shown in FIG. 11. That is, the nostrils brightness is considered light. When inhaling, the temperature of the nostrils is slightly cooler than the normal human body temperature as shown in FIG. 12. That is, the nostrils brightness is considered dark. Thus, when an adult completes one respiratory cycle—one inhalation followed by one exhalation—within 3-5 seconds, their breathing is considered abnormal, which is also indicated by regular changes in the nostrils' brightness, alternating between light and dark regularly. That is, if the nostrils' brightness remains unchanged during one respiratory cycle, their breathing is considered abnormal. the nostrils appears white within the window 20 in one of the continuous thermal images (THI) during exhalation, while the nostrils appears black within the window 20 in another one of the series of continuous thermal images (THI) during inhalation. Upon statistics, one experimental data can be presented in Table 3 as below.

TABLE 3

| | | Actual | Identify | | Omission | | | omission | |
|---|---|---|---|---|---|---|---|---|---|
| | Standard | breath | breath | difference | count | Error count | accuracy | rate | error rate |
| Avg. | manual | 21.9 | 20.5 | 1.4 | 1.2 | 0.2 | 95.68% | 3.51% | 1.23% |

According to a ground truth, YOLO can be trained to identify brightness changes in the nasal area of the infant sleeping during exhalation and inhalation. The ground truth can be the time point labelled in nostrils during exhalation. Additionally, the training data is divided into three sets, a training set, a validation set, and a test set. For instance, 40% of the series of continuous thermal images (THI) can be the training set, 10% of the series of continuous thermal images (THI) can be the validation set, and 50% of the series of continuous thermal images (THI) can be the test set.

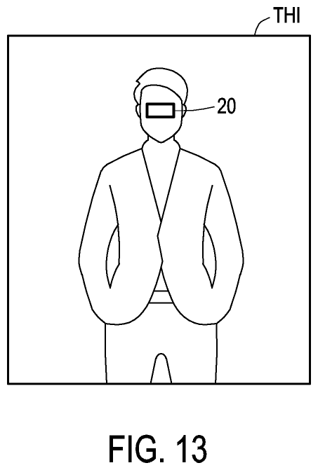

Pleas refers to FIGS. 4A, 4E, and 13, the process of the forehead area identification in the thermal images (THI) includes steps S150, S170, and S180-S187 described as below in detail.

In one embodiment, in step S180, when the thermal imaging sensor 104 in detector 100 faces the individual and detects the individual's temperature, the host computer 200 receives a plurality of temperatures readings detected from the forehead area in the current thermal images (THI). The forehead area in the current thermal images (THI) is identified by the real-time object detection algorithm, executed by the host computer 200. Next, in step S181, the host computer 200 can compensate the temperature error through forehead identification, constant temperature calibration, and distance calibration. Thus, in step S183, the host computer 200 determines whether the average temperature is within a normal temperature range from 34.7° C. to 37.5° C., wherein the physiological information further comprises forehead temperature. If YES, the generative method proceeds to step S185, which includes recording and storing the normal temperature information. If No, the generative method proceeds to step S187, which includes notifying the abnormal temperature information.

As shown in FIG. 13, YOLO can be trained to identify a forehead area in the current thermal images. After that, the detector is configured to detect a plurality of temperatures at different positions within the forehead area, and the host computer 200 is configured to calculate a temperature error between the measured temperature and a normal temperature for temperature compensation. The normal temperature is obtained from the average temperature of three different positions on a test subject's forehead area using an infrared forehead thermometer. The infrared forehead thermometer is approved by the Food and Drug Administration (FDA) of the United States. Based on the experimental data, a temperature error can be presented in Table 3 as below. Thus, the average error is 0.14° C.

TABLE 3

| | | | | | | Actual |
|---|---|---|---|---|---|---|
| | | | | | | forehead |
| count | Test 1 | Test 2 | Test 3 | Test 4 | Test 5 | temperature |
| 1. | 36.5 | 36.5 | 36.5 | 36.5 | 36.5 | 36.5 |
| 2. | 36.5 | 36.4 | 36.3 | 36.3 | 36.4 | 36.4 |
| 3. | 36.4 | 36.5 | 36.6 | 36.6 | 36.4 | 36.3 |
| 4. | 36.4 | 36.4 | 36.4 | 36.3 | 36.3 | 36.3 |
| 5. | 36.5 | 36.5 | 36.5 | 36.4 | 36.4 | 36.3 |
| 6. | 36.5 | 36.5 | 36.5 | 36.4 | 36.5 | 36.3 |
| 7. | 36.4 | 36.4 | 36.6 | 36.4 | 36.5 | 36.3 |
| 8. | 36.4 | 36.5 | 36.5 | 36.5 | 36.5 | 36.6 |
| 9. | 36.6 | 36.6 | 36..3 | 36.6 | 36.5 | 36.3 |
| 10. | 36.5 | 36.5 | 36.5 | 36.5 | 36.4 | 36.3 |
| 11. | 36.5 | 36.4 | 36.3 | 36.3 | 36.3 | 36.3 |
| 12. | 36.6 | 36.5 | 36.4 | 36.6 | 36.6 | 36.3 |
| 13. | 36.5 | 36.5 | 36.6 | 36.5 | 36.6 | 36.3 |
| 14. | 36.5 | 36.5 | 36.5 | 36.5 | 36.5 | 36.3 |
| 15. | 36.5 | 36.5 | 36.5 | 36.4 | 36.4 | 36.3 |
| 16. | 36.5 | 36.6 | 36.5 | 36.6 | 36.5 | 36.4 |
| 17. | 36.5 | 36.5 | 36.5 | 36.5 | 36.5 | 36.3 |
| 18. | 36.5 | 36.5 | 36.5 | 36.4 | 36.4 | 36.3 |
| 19. | 36.5 | 36.4 | 36.3 | 36.3 | 36.3 | 36.2 |
| 20. | 36.4 | 36.4 | 36.3 | 36.3 | 36.4 | 36.3 |

Additionally, the host computer 200 can calibrate the measured temperature using a thermometer that measures constant temperature. The thermal imaging sensor 104 in the detector 100 can continuously measure and track the temperature on an individual's forehead for error compensation. The thermal imaging sensor 104 in the detector 100 can also measure the temperature of multiple individuals simultaneously for the same place. The host computer 200 can correct the temperature error for the targeted individual by comparing their tracked temperature with the temperatures of surrounding individuals.

For example, when the host computer 200 executes the YOLO algorithm, YOLO can use the window 20 to identify a plurality of different positions within the forehead area and measures temperatures at each of the positions through a thermal imager. Next, the thermal imaging sensor 104 in the detector 100 can use the window 20 to measure the temperature on the forehead area in each thermal image (THI) after YOLO identifies the same forehead area in the same thermal images (THI). Thus, through software, the processor can compensate for the errors, and also through hardware, adjust the errors if the average value falls within the temperature range, such as above 34.7° C. and below 37.5° C.

It will be apparent to those skilled in the art that various modifications and variations can be made to the present disclosure. It is intended that the specification and examples be considered as exemplary embodiments only, with a scope of the disclosure being indicated by the following claims and their equivalents.

What is claimed is:

1. A facial recognition system, comprising:
a detector, comprising a visible light sensor, configured to capture one or more current visible images within a target area, and a thermal imaging sensor, configured to capture one or more current thermal images within the target area; and
a host computer, coupled to the detector and configured to execute a real-time object detection algorithm to notify abnormal respiratory information when the host computer determines a nasal area in the current thermal images is abnormal, wherein
the real-time object detection algorithm, configured to identify a face region in the current visible and thermal images, when the face region in the current visible images is not identifiable, the real-time object detection algorithm identifies the nasal area of the face region in the current thermal images, and the host computer generates the abnormal respiratory information according to the cycles of exhalation and inhalation, as detected through brightness changes in the nostril area.

2. The facial recognition system according to claim 1, wherein the host computer comprises receiving a plurality of temperatures readings detected from the brightness changes in the nostril area, comparing the temperature difference in the nostril area with a normal respiratory standard to determine that respiratory information is abnormal.

3. The facial recognition system according to claim 1, wherein the host computer comprises determining that respiratory information is abnormal when the brightness in the nostrils remains unchanged, exceeding the duration of a normal respiratory standard.

4. The facial recognition system according to claim 1, wherein the host computer comprises converting the number of times that brightness alternates between light and dark to an individual's breaths per minute (BPM), comparing the converted individual's breaths per minute with the criteria of the individual's breaths per minute, and when the converted individual's breaths per minute is above or below the criteria of the individual's breaths per minute, determining that the respiratory information is abnormal.

5. The facial recognition system according to claim 2, wherein, before detecting the brightness changes in the nostril area, alternating between light and dark in the current thermal images, the real-time object detection algorithm is configured to classify the nostril during inhalation and the nostril during exhalation.

6. The facial recognition system according to claim 1, wherein, when the face region in the current visible images is not identifiable, the host computer is configured to identify brightness changes of the forehead area, transform the brightness changes of the forehead area into a spectrum to extract at least one peak frequency as the heart rate information, and determine that the heart rate information is abnormal if the peak frequency is above or below a normal range.

7. The facial recognition system according to claim 6, wherein, when the face region in the current visible images is not identifiable, the host computer is configured to calculate the brightness changes of pixels within the forehead area, and transforms the pixels from the time domain into the frequency domain to generate the spectrum.

8. The facial recognition system according to claim 1, wherein, when the face region in the current visible images is not identifiable, the host computer is configured to identify both a mouth area and the nasal area in the current visible images, and then determine the respiratory information is abnormal if the mouth area or the nasal area are obscured by at least one cover.

9. The facial recognition system according to claim 1, wherein the real-time object detection algorithm is configured to identify the forehead area in the current thermal images; the detector is configured to detect a plurality of temperatures at different positions within the forehead area; and then the host computer is configured to receive and average a plurality of temperatures readings detected from the brightness changes in the nostril area and determine that the average temperature is abnormal if the average temperature is above or below a temperature range.

10. The facial recognition system according to claim 1, further comprising an alarm component, coupled to the processor to notify the abnormal respiratory information or the abnormal the heart rate information.

11. The facial recognition system according to claim 1, further comprising a communication component, coupled to the processor to transmit the physiological information on the internet.

12. A generative method for physiological information, executed by a processor in a facial recognition system using a real-time object detection algorithm, the generative method comprising:
receiving one or more current visible and thermal images from a detector in the facial recognition system;
identifying a nasal area in the current thermal images when a face region in the current visible images is not identified by the real-time object detection algorithm;
determining that respiratory information is abnormal according to the cycles of exhalation and inhalation, as detected through brightness changes in the nostril area; and
notifying abnormal respiratory information.

13. The generative method for physiological information according to claim 12, wherein the step of determining that respiratory information is abnormal further comprising: receiving a plurality of temperatures readings detected from the brightness changes in the nostril area, comparing the temperature difference in the nostril area with a normal respiratory standard to determine that respiratory information is abnormal.

14. The generative method for physiological information according to claim 12, wherein the step of determining that respiratory information is abnormal further comprising: when the brightness in the nostrils remains unchanged, exceeding the duration of a normal respiratory standard, the processor determines that respiratory information is abnormal.

15. The generative method for physiological information according to claim 12, wherein the step of determining that respiratory information is abnormal further comprising: converting the number of times that brightness alternates between light and dark to an individual's breaths per minute (BPM), comparing the converted individual's breaths per minute with the criteria of the individual's breaths per minute, and when the converted individual's breaths per minute is above or below the criteria of the individual's breaths per minute, determining that the respiratory information is abnormal.

16. The generative method for physiological information according to claim 13, wherein, before detecting the brightness changes in the nostril area, alternating between light and dark in the current thermal images, the real-time object detection algorithm is configured to classify the nostril during inhalation and the nostril during exhalation.

17. The generative method for physiological information according to claim 12, wherein, when the face region in the current visible images is not identifiable, the host computer is configured to identify brightness changes of the forehead area, transform the brightness changes of the forehead area into a spectrum to extract at least one peak frequency as the heart rate information, and determine that the heart rate information is abnormal if the peak frequency is above or below a certain range.

18. The generative method for physiological information according to claim 12, wherein, when the face region in the current visible images is not identifiable, the host computer is configured to identify both a mouth area and the nasal area in the current visible images, and then determine the respiratory information is abnormal if the mouth area or the nasal area are obscured by at least one cover.

19. The generative method for physiological information according to claim 12, wherein the real-time object detection algorithm is configured to identify the forehead area in the current thermal images; the detector is configured to detect a plurality of temperatures at different positions within the forehead area; and then the host computer is configured to receive and average a plurality of temperatures readings detected from the brightness changes in the nostril area and determine that the average temperature is abnormal if the average temperature is above or below a temperature range.
